(19) United States
(12) Patent Application Publication
(10) Pub. No.: US 2016/0074769 A1
(43) Pub. Date: Mar. 17, 2016

(54) Drum Trap Capable of Condensing/Circulating Vapor and Food Waste Treatment Apparatus Using the Same

(71) Applicants: Nou-Hyeong PARK, Gwangju-si (KR); Byeon-Sam KIM, Bucheon-si (KR)

(72) Inventors: Nou-Hyeong PARK, Gwangju-si (KR); Byeon-Sam KIM, Bucheon-si (KR)

(73) Assignee: SPINZ INNOVATION CO., LTD., Seongnam-si (KR)

(21) Appl. No.: 14/559,815

(22) Filed: Dec. 3, 2014

(30) Foreign Application Priority Data
Sep. 16, 2014 (KR) 10-2014-0122529

(51) Int. Cl. B01D 1/30; B01D 5/00; A61L 11/00

(52) U.S. Cl. CPC B01D 1/30; A61L 11/00; B01D 5/0003

(57) ABSTRACT

Disclosed are a drum trap and a food waste treatment apparatus using the same. In the drum trap according to the present invention and the food waste treatment apparatus using the same, the fetid vapor generated when drying food waste in the drying unit is introduced into the drum trap, condensed, and then discharged to the drain pipe side. In addition, the fetid vapor remaining without being condensed is re-introduced into the drying unit. That is, since the fetid vapor generated when drying the food waste does not leak out to the outside, no separate filter for removing the fetid smell contained in the vapor is required. Accordingly, production costs may be reduced.

### BACKGROUND OF THE INVENTION

1. Field of the Invention

The present invention relates to a drum trap capable of condensing and discharging fetid vapor, and circulating non-condensed fetid vapor, and a food waste treatment apparatus using the same.

2. Description of the Prior Art

Food waste is a factor in polluting the environment and the discharge amount of the food waste tends to increase every year.

Research and development for various types of food waste treatment apparatuses have been actively made to treat the food waste in an environmentally friendly manner.

The food waste treatment apparatuses may be generally classified into a drying-type food waste treatment apparatus which dries and treats cut and crushed food waste, and a fermenting-type waste treatment apparatus which ferments and treats cut and crushed food waste while storing the food waste for a predetermined length of time.

The drying-type food waste treatment apparatus dehydrates cut and crushed food waste and then removes water contained in the dehydrated food waste. Thus, the drying-type food waste treatment apparatus has an advantage in that the discharge amount of the food waste is considerably reduced.

However, the drying-type food waste treatment apparatus generates a fetid smell when drying the food waste. As a result, the drying-type food waste treatment apparatus should be provided with, for example, a filter to remove the fetid smell, and the filter should be replaced periodically. Therefore, there is a disadvantage in that the production costs increase.

A prior art related to a drying unit of a food waste treatment apparatus is disclosed in Korean Laid-Open Patent Publication No. 10-2009-0089834.

### SUMMARY OF THE INVENTION

An aspect of the present invention is to provide a drum trap capable of solving all the problems in the prior art as described above and a food waste treatment apparatus using the same.

Another aspect of the present invention is to provide a drum trap capable of condensing and discharging fetid vapor generated when drying food waste, and circulating fetid vapor remaining without being condensed, thereby reducing production costs, and a food waste treatment apparatus using the drum trap.

In accordance with an aspect, there is provided a drum trap including: a housing including an opened top surface and one side surface in which a discharge unit is formed to discharge water, the discharge unit being configured to store water in a bottom side inner portion thereof; and a cooling plate module coupled to the housing to condense vapor introduced into the housing. The cooling plate module may include: a first cooling plate coupled to the opened top surface of the housing; a second cooling plate formed to extend downwardly from a bottom surface of the first cooling plate and positioned inside of the housing, a bottom side portion of the second cooling plate being positioned below the discharge unit to be submerged in the water stored in the housing; and an introduction pipe formed to extend downwardly from a central portion of the first cooling plate so as to introduce water into the inside of the housing therethrough. An inlet hole and an outlet hole are formed in one side portion and the other side portion, respectively, so that vapor is introduced into the inside of the housing through the inlet hole, and non-condensed vapor in the vapor introduced into the inside of the housing through the inlet hole is discharged through the outlet hole.

According to the present invention, there is also provided a food waste treatment apparatus including: a drying drum configured to dry food waste put into the drying drum; and a drum trap configured to receive vapor generated when the food waste is dried in the drying drum, and condense and discharge the vapor. The drum trap may include: a housing including an opened top surface and one side surface in which a discharge unit is formed to discharge water to the drain pipe side, the discharge unit being configured to store water in a bottom side inner portion thereof; and a cooling plate module coupled to the housing to condense vapor introduced into the housing. The cooling plate module may include: a first cooling plate coupled to an opened top end surface of the housing in which one side and the other side of the first cooling plate are communicated with one side and the other side of the drying drum, respectively; a second cooling plate formed to extend downwardly from a bottom surface of the first cooling plate and positioned inside of the housing, a bottom side portion of the second cooling plate being positioned below the discharge unit to be submerged in the water stored in the housing; and an introduction pipe formed to extend downwardly from a central portion of the first cooling plate so as to introduce water into the inside of the housing therethrough. An inlet hole is formed at the one side of the cooling plate and an outlet hole is formed at the other side of the first cooling plate, in which the inlet hole is communicated with the one side of the drying drum so that the vapor generated when drying the food waste is introduced into the inside of the housing therethrough, and the outlet hole is communicated with the other side of the drying drum to guide non-condensed vapor in the vapor introduced into the inside of the housing through the inlet hole so that the non-condensed vapor is discharged to the drying drum.

In the drum trap according to the present invention and the food waste treatment apparatus using the same, the fetid vapor generated when drying food waste in the drying unit is introduced into the drum trap, condensed, and then discharged to the drain pipe side. In addition, the fetid vapor remaining without being condensed is re-introduced into the drying unit. That is, since the fetid vapor generated when drying the food waste does not leak out to the outside, no separate filter is required to remove the fetid smell contained in the vapor. Accordingly, production costs may be reduced.

In addition, since the fetid vapor generated when drying the food waste is condensed within the drum trap, it is not necessary to install a cooling pipe outside of the drum trap to condense the vapor. Accordingly, it is easy to install the drum trap in the food waste treatment apparatus, and to separate the drum trap from the food waste treatment apparatus. Therefore, maintenance may be conveniently performed.

In addition, since the bottom side portion of the cooling plate module of the drum trap is submerged in the water stored in the drum trap, the cooling plate module is always kept in the cold state. Accordingly, most of the vapor introduced into the drum trap may be condensed by the cooling plate module and then discharged.

In addition, the inside of the housing of the drum trap is partitioned into the inlet hole side and the outlet hole side, and the vapor introduced into the drum trap through the inlet hole is circulated in the drum trap and then discharged through the outlet hole. Thus, a vapor condensing efficiency can be improved.

In addition, since the bottom side portion of the elbow tube of the discharge unit of the drum trap is submerged in the water stored in the housing, the hot vapor introduced into the drum trap cannot be discharged to the drain pipe side. Therefore, the discharge pipe and a connecting pipe connecting the discharge pipe and the drain pipe side can be prevented from being damaged by vapor.

In addition, since the bottom side portion of the elbow tube of the discharge unit of the drum trap is submerged in the water stored in the drum trap, a fetid smell generated from the drain pipe side can be prevented from being introduced into the drum trap.

Further, since the vapor re-introduced into the drying unit through the outlet hole of the drum trap is dry vapor containing relatively less moisture, the moisture of the food waste put into the drying unit can be quickly absorbed. Accordingly, a drying efficiency can be enhanced.

### BRIEF DESCRIPTION OF THE DRAWINGS

The above and other objects, features and advantages of the present invention will be more apparent from the following detailed description taken in conjunction with the accompanying drawings, in which:

FIG. 1 is a perspective view of a food waste treatment apparatus according to an exemplary embodiment of the present invention;

FIG. 2 is a perspective view of a drying unit part and a drum trap part of the food waste treatment apparatus according to an exemplary embodiment of the present invention;

FIG. 3 is a perspective view illustrating the parts of FIG. 2, which are viewed from the right side;

FIG. 4 is an exploded perspective view of the drum trap part illustrated in FIG. 3;

FIG. 5 is a bottom side perspective view illustrating the drum trap part of FIG. 4, which is viewed in another direction;

FIG. 6 is a cross-sectional view taken along line A-A in FIG. 4;

FIG. 7 is a cross-sectional view taken along line B-B of FIG. 4 in the assembled state;

FIG. 8 is a front view of the valve illustrated in FIG. 7; and

FIG. 9 is a cross-sectional view taken along line C-C in FIG. 4 in the assembled state.

### DETAILED DESCRIPTION OF THE EXEMPLARY EMBODIMENTS

Hereinafter, embodiments of the present invention will be described in detail with reference to the accompanying drawings.

In the accompanying drawings, a portion irrelevant to a description of the present invention will be omitted for clarity. Like reference numerals refer to like elements throughout.

The terms described in the specification should be understood as follows.

Terms like a first and a second may be used to describe various elements, but the elements should not be limited by the terms. The terms may be used only as object for distinguishing an element from another element. For example, without departing from the spirit and scope of the inventive concept, a first element may be referred to as a second element, and similarly, the second element may be referred to as the first element.

In the following description, the technical terms are used only for explaining a specific exemplary embodiment while not limiting the present invention. The terms of a singular form may include plural forms unless referred to the contrary. The meaning of “include,” “comprise,” “including,” or “comprising,” specifies a property, a region, a fixed number, a step, a process, an element and/or a component but does not exclude other properties, regions, fixed numbers, steps, processes, elements and/or components.

Hereinafter, a drum trap according to an exemplary embodiment of the present disclosure and a food waste treatment apparatus using the same will be described in detail with reference to the accompanying drawings.

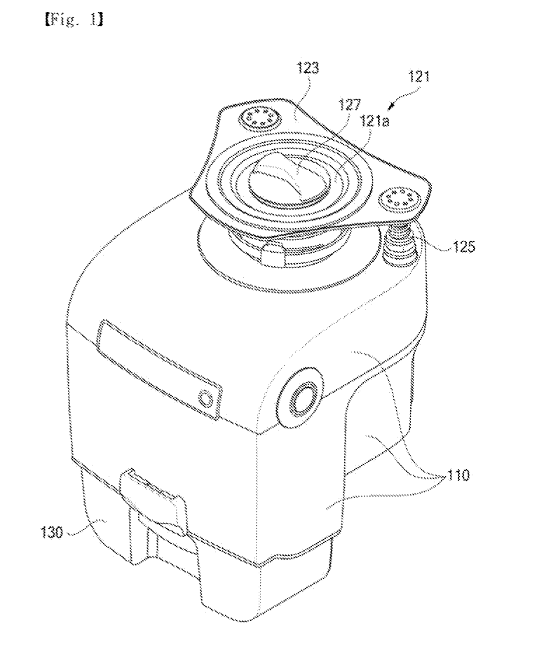

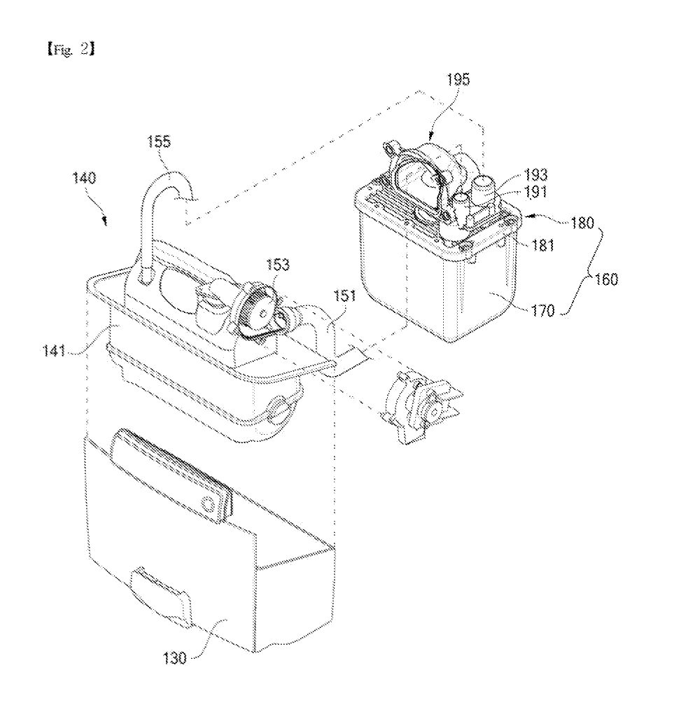

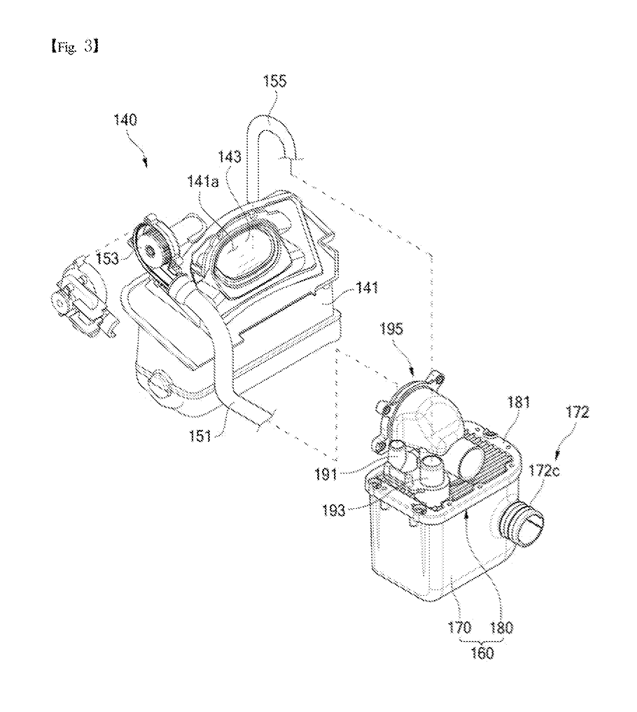

FIG. 1 is a perspective view of a food waste treatment apparatus according to an exemplary embodiment of the present disclosure, FIG. 2 is a perspective view of a drying unit part and a drum trap part of the food waste treatment apparatus of the exemplary embodiment of the present disclosure, and FIG. 3 is a perspective view illustrating the parts of FIG. 2, which are viewed from the right side.

As illustrated, the food waste treatment apparatus according to the present exemplary embodiment may be installed below a wash basin (not illustrated) of a sink (not illustrated), and may include a casing 110 forming an external appearance. The casing 110 may be formed by assembling an upper casing, an intermediate casing, and a lower casing with each other.

Hereinafter, in designating a surface and a direction of other constituent elements including the casing 110, a surface or a direction directed toward the top side of the casing 110 will be referred to as a “top surface” or a “top side” and a surface or a direction directed toward the bottom side of the casing 110 will be referred to as a “bottom surface” or a “bottom side”.

A connecting holder 121 may be installed at the top side of the casing 110, and coupled to and supported by the wash basing (not illustrated) of the sink (not illustrated). The connecting holder 121 is inserted into a drain aperture (not illustrated) formed on the bottom surface of the wash basin, and a fastening rim 121a formed on an outer circumferential surface of the top end side of the connecting holder 121 may be fastened to and supported by the bottom surface of the wash basin that forms the drain aperture.

A support plate 123 may be installed on and supported by the outer circumferential surface of the connecting holder 121. When the connecting holder 121 is inserted into and coupled to the wash basin, the peripheral edge side of the support plate 123 may be contacted with and supported by the bottom surface of the wash basin.

A buffering/leveling unit 125 may be installed between the top surface of the casing 110 and the support plate 123, in which the buffering/leveling unit 125 is configured to buffer and transfer vibration of the food waste treatment apparatus to the support plate 123 as well as to level the casing 110 with respect to the sink.

A cutting/crushing unit (not illustrated) may be installed at the top side of an inner rear surface of the casing 110 in which the cutting/crushing unit is configured to cut and crush food waste put into the connecting holder 121 through the drain aperture of the wash basin. A dehydrating unit (not illustrated) may be installed below the cutting/crushing unit in which the dehydrating unit is configured to receive and dehydrate the cut and crushed food waste.

The dehydrating unit includes a drain aperture (not illustrated), and the water discharged through the drain aperture of the dehydrating unit may be introduced into a drum trap 160 to be described later.

The configurations of the cutting/crushing unit and the dehydrating unit may be the same as or similar to those in the food waste treatment apparatus disclosed in Korean Patent No. 10-1104573 filed and issued in the name of Nou Hyeong PARK who is an inventor of the present application.

The dehydrating unit may dehydrate the food waste using centrifugal force, and discharge the dehydrated food waste using the centrifugal force. That is, the dehydrated food waste may be discharged to a drying unit 140 through a portion where the centrifugal force of the dehydrating unit is strongest, and the drying unit 140 may dry the dehydrated food waste.

As illustrated in FIGS. 2 and 3, the drying unit 140 may include a drying drum 141.

An inlet port 141a may be formed in the top surface of the drying drum 141 to introduce the food waste dehydrated in the dehydrating unit therethrough, in which the inlet port 141a may be opened/closed by an opening/closing member 143 which is installed to be rotatable. When the inlet port 141a is opened by the opening/closing member 143, the food waste ejected from the dehydrating unit may be put into the inside of the drying drum 141 through the inlet port 141a.

An outlet port (not illustrated) may be formed in the bottom surface of the drying drum 141 to discharge dried food waste therethrough, in which the outlet port of the drying drum 141 may also be opened/closed by an opening/closing member (not illustrated) which is similar to the opening/closing member 143. A heating unit (not illustrated) configured to heat the dehydrated food waste, such as a heater, a hot air blower, or a microwave generator, may be installed on the outer surface of the drying drum 141 or inside of the drying drum 141.

An agitating screw (not illustrated) may be rotatably installed within the drying drum 141 to agitate the food waste while the food waste is dried within the drying drum 141. The agitating screw may enhance a drying efficiency by agitating the food waste, and may crush the food waste when the food waste is congealed and tends to be solidified to be hard like a stone.

When the food waste is dried, fetid vapor is generated. When the fetid vapor is discharged to the outside, a user may feel inconvenience and the environment may be polluted.

The drum trap according to the present exemplary embodiment and the food waste treatment apparatus using the same may be configured to condense and discharge the fetid vapor generated when drying the food waste to a drain pipe side, and to re-introduce the fetid vapor remaining without being condensed into the drying drum 141.

Specifically, one end sides of a first guide pipe 151 and a second guide pipe 155 may be installed to be communicated with one side and the other sides of the top surface of the drying drum 141, respectively, and the other end side of the first guide pipe 151 and the second guide pipe 155 may be installed to be communicated with one side and the other side within the drum trap 160, respectively. The drum trap 160 will be described later.

The fetid vapor generated when drying the food waste in the drying drum 141 may be introduced into the inside of the drum trap 160 through the first guide pipe 151, and the fetid vapor remaining without being condensed within the drum trap 160 may be re-introduced into the drying drum 141 through the second guide pipe 155.

In order to cause the vapor in the drying drum 141 to be quickly introduced into the first guide pipe 151, a fan 153 may be installed inside of one side of the first guide pipe 151 which is adjacent to the drying drum 141 so as to forcibly suck the vapor within the drying drum 141 into the first guide pipe 151.

One side of the inside of the drum trap 160 may also be connected with the drain pipe side, and the water introduced into the inside of the drum trap 160 and condensed may be discharged to the drain pipe side.

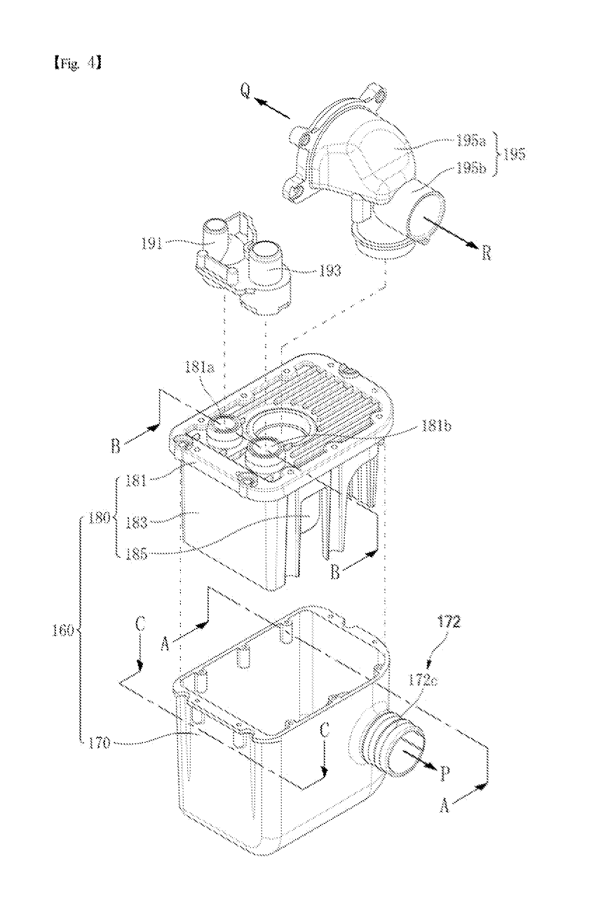

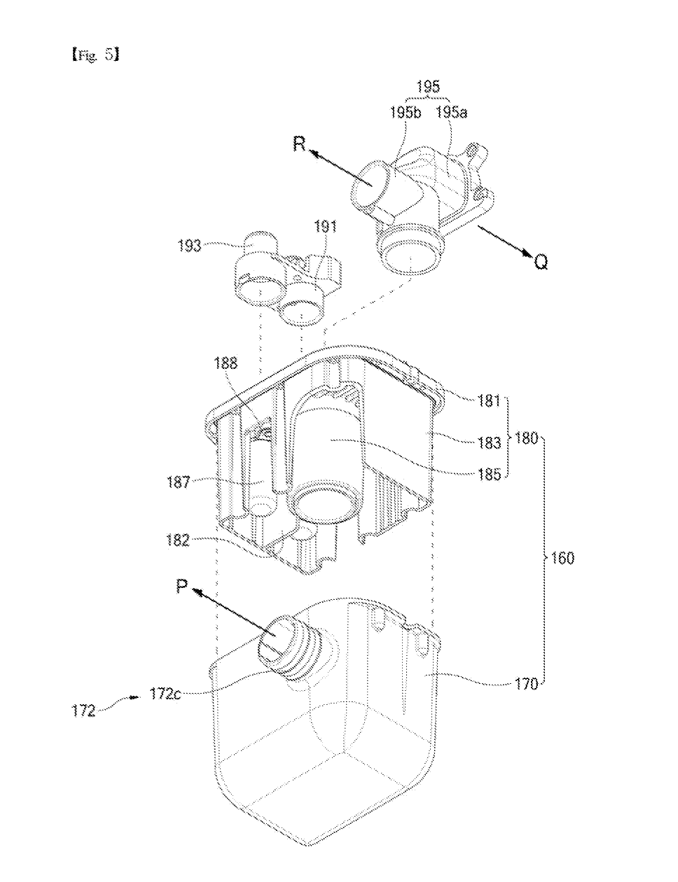

The drum trap 160 will be described with reference to FIGS. 2 to 6. FIG. 4 is an exploded perspective view of the drum trap part illustrated in FIG. 3, FIG. 5 is a bottom side perspective view illustrating the drum trap part of FIG. 4, which is viewed in another direction, and FIG. 6 is a cross-sectional view taken along line A-A in FIG. 4.

As illustrated, the drum trap 160 may be formed in a case shape with a space formed therein, and may include a housing 170 formed of a synthetic resin material, and a cooling plate module 180 coupled to the top surface of the housing 170 and formed of a metallic material which is excellent in heat conductivity, such as aluminum.

The housing 170 may be top-opened, and a discharge unit 172 may be formed at one side of the housing 170 to discharge water. Water is stored within the housing 170, in which the water may be stored only below the discharge unit 172 within the housing 170. That is, when water is introduced into the housing 170, the water positioned below the discharge unit 172 may be stored in the housing 170, and the water positioned above the discharge unit 172 may be discharged to the drain pipe side through the discharge unit 172.

As illustrated in FIG. 6, the discharge unit 172 may include a through-hole 172a, an elbow tube 172b, and a discharge pipe 172c.

The through-hole 172a may be formed on a side surface of the housing 170 to communicate the inside and outside of the housing 170 with each other. The elbow tube 172b may be communicated with the through-hole 172a and protrude to the inside of the housing 170, in which the bottom side portion of the elbow tube 172b may be positioned below the through-hole 172a to be submerged in the water stored in the housing 170. In addition, the discharge pipe 172c may be communicated with the elbow tube 172b and protrude to the outside of the housing 170 to be communicated with the drain pipe side (in a direction indicated by arrow P in FIGS. 4 and 5).

Since the bottom side portion of the elbow tube 172b is submerged in the water, the vapor introduced into the housing 170 of the drum trap 160 is prevented from being discharged to the drain pipe side. Therefore, the discharge pipe 172c and a connecting pipe (not illustrated), which connects the discharge pipe 172c and the drain pipe side can be prevented from being damaged by hot vapor.

The cooling plate module 180 may include a first cooling plate 181, a second cooling plate 183, and an introduction pipe 185, and condense the vapor introduced into the housing 170.

The first cooling plate 181 may be formed in a shape corresponding to the top surface of the housing 170, and the bottom surface of the first cooling plate 181 may be coupled to be in contact with the opened top end surface of the housing 170. In addition, an inlet hole 181a may be formed in one side portion the first cooling plate 181 and an outlet hole 181b may be formed in the other side portion of the first cooling plate 181, in which the inlet hole 181a is communicated with the other end side of the first guide pipe 151 communicated with the drying drum 141 and the outlet hole 181b is communicated with the other end side of the second guide pipe 155 communicated with the drying drum 141.

Therefore, the fetid vapor in the drying drum 141 is introduced into the inside of the drum trap 160 through the first guide pipe 151 and the inlet hole 181a, and the fetid vapor remaining without being condensed may be re-introduced into the drying drum 141 through the outlet hole 181b and the second guide pipe 155. Accordingly, the fetid vapor generated when drying the food waste may be circulated between the drying drum 141 and the drum trap 160 without being discharged to the outside.

The bottom side portions of the connecting pipes 191 and 193 may be installed to be communicated with the inlet hole 181a and the outlet hole 181b, respectively, and the other end sides of the first guide pipe 151 and the second guide pipe 155 may be coupled to and communicated with the top side portions of the connecting pipe 191 and 193, respectively.

The second cooling plate 183 is formed to extend downwardly from the bottom surface of the peripheral edge side of the first cooling plate 181, and the bottom side portion of the second cooling plate 183 may be positioned below the through-hole 172a of the discharge unit 172 to be submerged in the water stored in the housing 170. Since the bottom side portion of the second cooling plate 183 is submerged in the water, the second cooling plate 183 and the first cooling plate 181 may be maintained in a cold state having a temperature which is similar to that of the water. Then, the vapor introduced into the drum trap 160 may exchange heat with the cold cooling plate module 180, thereby being quickly condensed.

The second cooling plate 183 may be inserted into the housing 170 to be in contact with the inner surface of the housing 170. Since the second cooling plate 183 made of the metallic material is inserted and coupled to be in contact with the inner surface of the housing 170 made of the synthetic resin material, the rigidity of the housing 170 may be reinforced.

The introduction pipe 185 may be formed to extend downwardly from a central portion of the first cooling plate 181 and supply water to the inside of the housing 170 through the introduction pipe 185. At this time, the bottom side portion of the introduction pipe 185 may also be positioned below the through-hole 172a of the discharge unit 172 to be submerged in the water stored in the housing 170. Then, since the first cooling plate 181 may be cooled more quickly due to the introduction pipe 185, the vapor introduced into the housing 170 may be condensed more quickly.

The water introduced into the housing 170 through the introduction pipe 185 may be the water discharged from the drain aperture of the dehydrating unit and the water discharged from an anti-overflow discharge hole (not illustrated) formed in a side surface of the wash basin of the sink.

The bottom side portion of the connecting pipe 195 may be installed to be communicated with the upper end side of the introduction pipe 185 so that the water discharged from the drain aperture of the dehydrating unit and the water discharged from the anti-overflow discharge hole may flow into the housing 170. A first branch pipe 195a and a second branch pipe 195b may be formed in the top side portion of the connecting pipe 195, and the first branch pipe 195a and the second branch pipe 195b may be communicated with the drain aperture side (in the direction indicated by arrow Q in FIGS. 4 and 5) and the anti-overflow discharge hole side (in the direction indicated by arrow R in FIGS. 4 and 5), respectively.

When the water at the drain pipe side flows backward to the inside of the drum trap 160 or the drain pipe side is blocked, the water in the drum trap 160 may flow into the drying unit 140 side. In order to prevent this, a valve 187 configured to prevent the water in the drum trap 160 from flowing into the drying unit 140 side may be installed in the drum trap according to the present exemplary embodiment and the food waste treatment apparatus using the same.

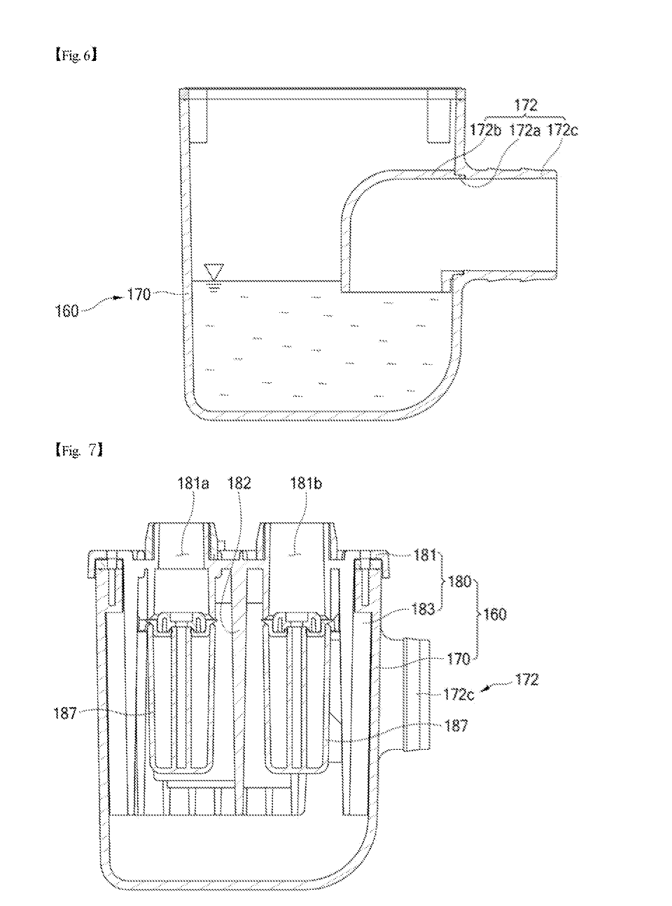

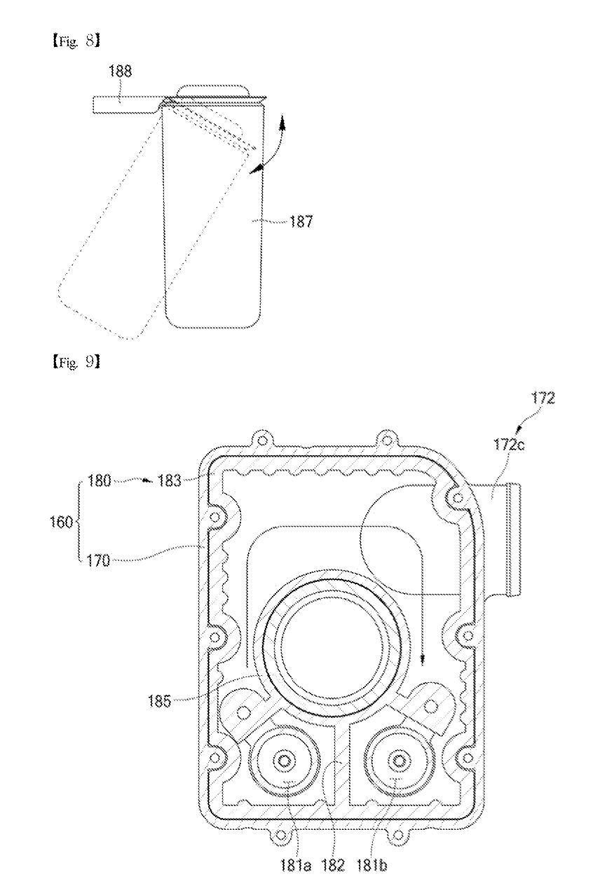

The valve 187 will be described with reference to FIGS. 7 and 8. FIG. 7 is a cross-sectional view taken along line B-B of FIG. 4 in the assembled state, and FIG. 8 is a front view of the valve illustrated in FIG. 7.

As illustrated, support pieces 188 are coupled to the bottom surface of the first cooling plate 181 at the inlet port 181a side and at the outlet port 181b, respectively, the upper end side outer circumferential surfaces of valves 187 may be connected to and supported by the end sides of the support pieces 188, respectively. In addition, the valves 187 may be installed to be rotatable about a portion where they are supported on the support pieces 188.

As such, the valves 187 may open/close the inlet hole 181a and the outlet hole 181b, respectively, while being rotated and moved up/down according to a level of water introduced into the housing 170. Each of the valves 187 may be formed as a hollow body so that the valves 187 may be easily rotated and moved up/down depending on the level of the housing 170. In addition, in order to ensure that the valves 187 may rigidly close the inlet hole 181a and the outlet hole 181b, the top side portions of the valves 187 may be provided in a form of enclosing the outside of the inlet hole 181a and the outlet hole 181b to be in close contact with the bottom surfaces of the first cooling plate 181, or to be inserted into the inlet hole 181a and the outlet hole 181b. When the water level in the housing 170 rises, the top side portions of the valves 187 may be in more close contact with the first cooling plate 181, or further inserted into the inlet hole 181a and the outlet hole 181b so that the inlet hole 181a and the outlet hole 181b may be further sealed.

Then, even if the water level in the housing 170 rises by, for example, backflow of the drain pipe side water to the housing 170, the valves 187 may close the inlet hole 181a and the outlet hole 181b while being moved up and rotated so that the water in the housing 170 may be prevented from flowing into the drying unit 140 side.

When vapor introduced into the housing 170 through the inlet hole 181a stays inside of the housing 170 for an increased length of time, a heat exchanging time between the cooling plate module 180 and the vapor increases so that more vapor may be condensed.

The drum trap according to the present exemplary embodiment and the food waste treatment apparatus using the same may be configured as follows. The inlet hole 181a side space of the hosing 170 and the outlet hole 181b side space of the housing 170 may be partitioned so as to allow the vapor introduced through the inlet hole 181a to be circulated in the housing 170 and then discharged through the outlet hole 181b so that the vapor introduced into the housing 170 may stay within the housing 170 for a long time.

The configuration causing the vapor introduced through the inlet hole 181a to be circulated in the housing 170 and then discharged through the outlet hole 181b will be described with reference to FIGS. 7 and 9. FIG. 9 is a cross-sectional view taken along line C-C in FIG. 4 in the assembled state.

As illustrated, a partition plate 182 may be formed to extend downward on the bottom surface of the first cooling plate 181. At this time, the partition plate 182 is positioned between one side portion of the second cooling plate 183 and one side portion of the introduction pipe 185 and may be formed integrally with the first cooling plate 181, the second cooling plate 183, and the introduction pipe 185. In addition, the partition plate 182 may be positioned between one side surface of the housing 170 where the discharge unit 172 is formed and the other side surface of the housing 170 which faces the discharge unit 172, and the bottom side portion of the partition plate 182 may be positioned below the discharge unit 172 to be submerged in the water stored in the housing 170.

Then, in the space of the housing 170 above the water store in the housing 170, the space of the housing 170 where the partition plate 182 is positioned may be partitioned by the partition plate 182 and the introduction pipe 185. At this time, the inlet hole 181a may be formed in the portion of the first cooling plate 181 positioned between the partition plate 182 and the other side surface of the housing 170 which faces the discharge unit 172, and the outlet hole 181b may be formed in the portion of the first cooling plate 181 positioned between the partition plate 182 and the one side surface of the housing where the discharge unit 172 is formed.

Due to this, the vapor introduced into the housing 170 through the inlet hole 181a is moved to the introduction pipe 185 side where the partition plate 182 is not formed, and then discharged through the outlet hole 181b, as indicated by an arrow in FIG. 9. Accordingly, the vapor introduced into the housing 170 through the inlet hole 181a may be circulated in the housing 170 and then discharged through the outlet hole 181b.

Since the bottom side portion of the partition plate 182 is submerged in the water, the first cooling plate 181 may be cooled more quickly. Due to this, the vapor introduced into the drum trap 160 may be quickly condensed.

Of course, the drum trap 160 including the housing 170 and the cooling plate module 180 according to the present exemplary embodiment may be used by being installed in an apparatus which generates fetid vapor, besides the food waste treatment apparatus.

In addition, the food waste treatment apparatus according to the present exemplary embodiment may be configured such that food waste may be directly put into the drying unit 140 to be dried in a state where the waste is not crushed and dehydrated. At this time, the food waste may be crushed by the agitating screw of the drying unit 140.

In the drum trap according to the present exemplary embodiment and the food waste treatment apparatus using the same, the fetid vapor generated when drying food waste in the drying unit 140 is introduced into the drum trap 160, then condensed, and then discharged to the drain pipe side. In addition, the fetid vapor remaining without being condensed is re-introduced into the drying unit 140. That is, since the fetid vapor generated when drying the food waste does not leak out to the outside, no separate filter for removing the fetid smell contained in the vapor is required. Accordingly, production costs may be reduced.

In addition, since the vapor generated when drying the food waste is condensed within the drum trap 160, it is not necessary to install a cooling pipe outside of the drum trap 160 to condense the vapor. Accordingly, it is easy to install the drum trap 160 in the food waste treatment apparatus, and to separate the drum trap 160 from the food waste treatment apparatus. Accordingly, maintenance may be conveniently performed.

In addition, since the bottom side portion of the drum trap 160 is submerged in the water stored in the housing 170, the cooling plate module 180 always remains in the cold state. Accordingly, most of the vapor introduced into the housing 170 may be condensed by the cooling plate module 180 and discharged.

In addition, the inside of the housing 170 of the drum trap 160 is partitioned into the inlet hole 181a side and the outlet hole 181b side, and the vapor introduced into the housing 170 through the inlet hole 181a is circulated in the housing 170 and then discharged through the outlet hole 181b. Thus, a vapor condensing efficiency can be improved.

In addition, since the bottom side portion of the elbow tube 172b of the discharge unit 172 of the drum trap 160 is submerged in the water stored in the housing 170, the vapor introduced into the housing 170 cannot be discharged to the drain pipe side. Therefore, it is possible to prevent the discharge pipe 172c of the discharge unit 172, and a connecting pipe, which connects the discharge pipe 172c and the drain pipe side, from being damaged by vapor.

In addition, since the bottom side portion of the elbow tube 172b of the discharge unit 172 of the drum trap 160 is submerged in the water stored in the housing 170, a fetid smell generated from the drain pipe side can be prevented from being introduced into the drum trap 160.

Further, since the vapor re-introduced into the drying unit 140 through the outlet hole 181b of the drum trap 160 is dry vapor containing relatively less moisture, the moisture of the food waste put into the drying unit 140 can be quickly absorbed. Accordingly, a drying efficiency can be enhanced.

Reference numeral 127 not described in FIG. 1 denotes a plug opening/closing the connecting holder 121 exposed to the top side of the sink, in which the plug 127 may also function as an ON/OFF switch of the food waste treatment apparatus. In addition, reference numeral 130 not described in FIG. 1 denotes a food waste basket configured to store food waste discharged through the outlet port of the drying unit 140.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A drum trap comprising:
a housing including an opened top surface and one side surface in which a discharge unit is formed to discharge water, the discharge unit being configured to store water in a bottom side inner portion thereof; and
a cooling plate module coupled to the housing to condense vapor introduced into the housing,
wherein the cooling plate module comprises:
a first cooling plate coupled to the opened top surface of the housing;
a second cooling plate formed to extend downwardly from a bottom surface of the first cooling plate and positioned inside of the housing, a bottom side portion of the second cooling plate being positioned below the discharge unit to be submerged in the water stored in the housing; and
an introduction pipe formed to extend downwardly from a central portion of the first cooling plate so as to introduce water into the inside of the housing therethrough,
wherein an inlet hole and an outlet hole are formed in one side portion and the other side portion, respectively, so that vapor is introduced into the inside of the housing through the inlet hole, and non-condensed vapor in the vapor introduced into the inside of the housing through the inlet hole is discharged through the outlet hole.

2. The drum trap of claim 1, wherein the cooling plate module is made of a metallic material, and the housing is made of a synthetic material, and
the second cooling plate is coupled to be in contact with an inner surface of the housing.

3. The drum trap of claim 1, wherein a partition plate is formed on one side of the bottom surface of the first cooling plate to extend downwardly and positioned between the second cooling plate and the introduction pipe,
the partition plate is formed integrally with the first cooling plate, the second cooling plate, and the introduction pipe, and positioned between the one side surface of the housing where the discharge unit is formed and other side surface of the housing which faces the discharge unit,
each of a bottom side portion of the partition plate and a bottom side portion of the introduction pipe is positioned below the discharge unit to be submerged in the water stored in the housing, and
in a space of the housing above the water stored in the housing, a space of the housing where the partition plate is positioned is partitioned by the partition plate, the second cooling plate, and the introduction pipe.

4. The drum trap of claim 3, wherein the inlet hole is formed in a portion of the first cooling plate positioned between the partition plate and the other side surface of the housing which faces the discharge unit, and
the outlet hole is formed in a portion of the first cooling plate positioned between the partition plate and the one side surface of the housing where the discharge unit is formed.

5. The drum trap of claim 1, wherein the discharge unit includes:
a through-hole formed in the one side surface of the housing;
an elbow tube communicated with the through-hole and protruding to the inside of the housing, a bottom side portion of the elbow tube being positioned below the through-hole to be submerged in the water stored in the housing; and
a discharge pipe communicated with the elbow tube and protruding to the outside of the housing.

6. The drum trap of claim 1, wherein valves are provided inside of the housing to open/close the inlet hole and the outlet hole, respectively.

7. The drum trap of claim 6, wherein support pieces are coupled to the bottom surface of the first cooling plate at the inlet hole side and the outlet hole side, respectively,
one sides of the valves are connected to and supported by the support pieces, respectively, and
the valves are installed to be rotatable about portions supported by the support pieces so that the valves open/close the inlet hole and the outlet hole, respectively, while being rotated and moved up/down depending on a water level within the housing.

8. A food waste treatment apparatus comprising:
a drying drum configured to dry food waste put into the drying drum; and
a drum trap configured to receive vapor generated when the food waste is dried in the drying drum, and condense and discharge the vapor,
wherein the drum trap comprises:
a housing including an opened top surface and one side surface in which a discharge unit is formed to discharge water to the drain pipe side, the discharge unit being configured to store water in a bottom side inner portion thereof; and
a cooling plate module coupled to the housing to condense vapor introduced into the housing,
wherein the cooling plate module comprises:
a first cooling plate coupled to the opened top surface of the housing, one side and the other side of the first cooling plate being communicated with one side and the other side of the drying drum, respectively;
a second cooling plate formed to extend downwardly from a bottom surface of the first cooling plate and positioned inside of the housing, a bottom side portion of the second cooling plate being positioned below the discharge unit to be submerged in the water stored in the housing; and
an introduction pipe formed to extend downwardly from a central portion of the first cooling plate so as to introduce water into the inside of the housing therethrough,
wherein an inlet hole is formed at the one side of the first cooling plate and an outlet hole is formed at the other side of the first cooling plate, the inlet hole being communicated with the one side of the drying drum so that the vapor generated when drying the food waste is introduced therein, and the outlet hole being communicated with the other side of the drying drum to guide non-condensed vapor in the vapor introduced into the housing through the inlet hole so that the non-condensed vapor is discharged to the drying drum.

9. The food waste treatment apparatus of claim 8, wherein the cooling plate module is made of a metallic material, and the housing is made of a synthetic material, and
the second cooling plate is coupled to be in contact with an inner surface of the housing.

10. The food waste treatment apparatus of claim 8, wherein a partition plate is formed on one side of the bottom surface of the first cooling plate to extend downwardly and positioned between the second cooling plate and the introduction pipe,
the partition plate is formed integrally with the first cooling plate, the second cooling plate, and the introduction pipe and positioned between the one side surface of the housing where the discharge unit is formed and the other side surface of the housing which faces the discharge unit,
each of a bottom side portion of the partition plate and a bottom side portion of the introduction pipe is positioned below the discharge unit to be submerged in the water stored in the housing, and
in a space of the housing above the water stored in the housing, a space of the housing where the partition plate is positioned is partitioned by the partition plate, the second cooling plate, and the introduction pipe.

11. The food waste treatment apparatus of claim 10, wherein the inlet hole is formed in a portion of the first cooling plate positioned between the partition plate and the other side surface of the housing which faces the discharge unit, and
the outlet hole is formed in a portion of the first cooling plate positioned between the partition plate and the one side surface where the discharge unit is formed.

12. The food waste treatment apparatus of claim 8, wherein the discharge unit includes:
a through-hole formed in the one side surface of the housing;
an elbow tube communicated with the through-hole and protruding to the inside of the housing, a bottom side portion of the elbow tube being positioned below the through-hole to be submerged in the water stored in the housing; and
a discharge pipe communicated with the elbow tube to protrude to the outside of the housing and communicated with the drain pipe side.

13. The food waste treatment apparatus of claim 8, wherein valves are provided inside of the housing to open/close the inlet hole and the outlet hole, respectively.

14. The food waste treatment apparatus of claim 13, wherein support pieces are coupled to the bottom surface of the first cooling plate at the inlet hole side and the outlet hole side, respectively,
one sides of the valves are connected to and supported by the support pieces, respectively, and
the valves are installed to be rotatable about portions supported by the support pieces so that the valves open/close the inlet hole and the outlet hole, respectively, while being rotated and moved up/down depending on a water level within the housing.

15. The food waste treatment apparatus of claim 8, further comprising:
a dehydrating unit configured to dehydrate the food waste and put the dehydrated food waste into the drying unit,
wherein a connecting pipe is coupled to the introduction pipe to be communicated therewith, the connecting pipe including a first branch pipe and a second branch pipe being communicated with the dehydrating unit side and an anti-overflow discharge hole of a wash basin of a sink, respectively.
